(19) United States
(12) Patent Application Publication
(10) Pub. No.: US 2018/0369471 A1
(43) Pub. Date: Dec. 27, 2018

(54) DIALYSIS DEVICE

(71) Applicant: FRESENIUS MEDICAL CARE DEUTSCHLAND GMBH, Bad Homburg (DE)

(72) Inventor: Reiner SPICKERMANN, Wasserlosen-Burghausen (DE)

(73) Assignee: FRESENIUS MEDICAL CARE DEUTSCHLAND GMBH, Bad Homburg (DE)

(21) Appl. No.: 15/776,614

(22) PCT Filed: Nov. 17, 2016
(86) PCT No.: PCT/EP2016/001946

§ 371 Date: May 16, 2018

(30) Foreign Application Priority Data
Nov. 17, 2015 (DE) 10 2015 014 859.1

(51) Int. Cl. A61M 1/16; A61M 1/36

(52) U.S. Cl. CPC A61M 1/1664; A61M 2205/3673; A61M 2205/3606; A61M 1/3672

(57) ABSTRACT

The invention relates to a dialysis device having an extracorporeal blood system, a dialyzing fluid system, a dialyzer and a control unit, wherein the dialysis device has a heating mechanism for heating the blood in the extracorporeal blood system before entry into the dialyzer or in the dialyzer as well as a cooling mechanism for cooling the blood in the extracorporeal blood system after exiting the dialyzer and wherein the control unit is configured such that the blood is heated before entry into the dialyzer or in the dialyzer to a dialysis temperature which is above the body temperature of the patient and is cooled back to the body temperature of the patient after exiting the dialyzer.

The invention relates to a dialysis device having an extracorporeal blood system, a dialyzing fluid system, a dialyzer and a control unit.

Dialysis devices in accordance with the preamble of claim 1 are known from the prior art. Such devices are used within the course of a dialysis treatment to remove urea and other substances from the blood of a patient having no or reduced renal activity. The central element of a dialysis device is the dialyzer, with it being a filter unit having a blood chamber and a dialyzing fluid chamber which are separated by a semipermeable membrane. The substances to be removed from the blood pass through the semipermeable membrane from the blood chamber into the dialyzing blood chamber in the dialyzer.

To increase the efficiency of the treatment, it is desirable to achieve a throughput of the unwanted substances from the blood chamber into the dialyzing fluid chamber which is as high as possible.

Against the background, the invention relates to a dialysis device having an extracorporeal blood system, a dialyzing fluid system, a dialyzer and a control unit. In accordance with the invention, the dialysis device has a heating mechanism for heating the blood in the extracorporeal blood system before entry into the dialyzer or in the dialyzer as well as a cooling mechanism for cooling the blood in the extracorporeal blood system after exiting the dialyzer. The control unit is configured such that the blood is heated before entry into the dialyzer or in the dialyzer to a dialysis temperature which is above the body temperature of the patient and is cooled to the body temperature of the patient again after exiting the dialyzer.

The invention makes use of the finding that an increase in the blood temperature in the dialyzer has a positive effect on the purification performance in the dialyzer. The increasing of the blood temperature can in this respect take place by different apparatus outside the body. It is essential for the safety of the patient that the blood flowing back again has body temperature (within a certain tolerance range).

The temperature increase influences the purification performance in the dialyzer due to a plurality of effects. For example, the diffusion increases as the temperature increases, while the viscosity of the blood is reduced. Furthermore, at an elevated temperature, the balance between bound toxins, e.g. albumin-bound toxins which cannot permeate the membrane and free toxins which can permeate the membrane, is displaced in the direction of the free toxins. These effects combine together and produce a significantly higher purification performance overall.

In an embodiment, the dialysis temperature is between 37° C. and 46° C., preferably between 40° C. and 46° C., and further preferably at between 42° C. and 45° C. At temperatures beyond the limit of approximately 46° C., denaturation phenomena and other unwanted effects can begin.

In an embodiment, the heating mechanism comprises a heating apparatus arranged at the feed side of the dialyzer in the dialyzing fluid system. In this embodiment, the control unit is configured such that the dialyzing fluid is heated before entry into the dialyzer to a temperature which is larger than or at least equal to the dialysis temperature. The blood can thus be heated to the dialysis temperature by heat exchange in the dialyzer. The degree of the heating caused by the heat exchange can take place, with a given type of construction of the dialyzer, by a regulation of the flows of the dialyzing fluid and of the blood in the dialyzer and by a setting of the temperature of the dialyzing fluid.

In an embodiment, the dialysis device comprises a substitution fluid system which comprises a pre-dilution line opening into the extracorporeal blood system at the feed side of the dialyzer and/or a post-dilution line opening into the extracorporeal blood system at the return side of the dialyzer.

In an embodiment, the heating mechanism comprises a heating apparatus arranged at the feed side of the opening of the pre-dilution line in the substitution fluid system. In this embodiment, the control unit is configured such that the substitution fluid is heated before entry into the extracorporeal blood system through the pre-dilution line to a temperature which is larger than or at least equal to the dialysis temperature. The blood can thus be heated to the dialysis temperature before entry into the dialyzer by the addition of a hot substitution fluid. The degree of the heating can take place by a regulation of the flows of the substitution fluid and of the blood as well as by a setting of the temperature of the substitution fluid.

In an embodiment, the substitution fluid system is integrated into the dialyzing fluid system and the heating apparatus for the dialyzing fluid and for the substitution fluid can correspond to one another.

In an embodiment, the cooling mechanism comprises a cooling apparatus arranged at the feed side of the opening of the post-dilution line in the substitution fluid system. In this embodiment, the control unit is configured such that the substitution fluid is cooled before entry into the extracorporeal blood system through the post-dilution line to a temperature which is less than or at a maximum equal to the body temperature.

In an embodiment, the cooling mechanism comprises a branch for substitution fluid which is arranged at the feed side of the heating apparatus in the substitution fluid system and whose temperature is less than or at a maximum equal to the body temperature. The control unit is configured in this embodiment such that the substitution fluid is branched off before entry into the heating apparatus and is not heated up to body temperature before entry into the extracorporeal blood system through the post-dilution line.

Separate substitution fluid systems for pre-dilution and post-dilution can also be present instead of the branching.

The blood can thus again be cooled from the dialysis temperature to body temperature after exiting the dialyzer by the addition of a cool substitution fluid. The degree of the cooling can take place by a regulation of the flows of the substitution fluid and of the blood as well as by a setting of the temperature of the substitution fluid.

In an embodiment, the heating mechanism comprises a heating apparatus arranged at the feed side of the dialyzer in the blood system. In an embodiment, the cooling mechanism comprises a cooling apparatus arranged at the return side of the dialyzer in the blood system. A direct heating or cooling of the blood can thus take place.

The increase in the blood temperature is substantially possible by indirect heaters (e.g. flow heaters), by introducing heated fluid (hot pre-dilution); heated citrate is also conceivable with CiCa anticoagulation) or by heat exchange in the dialyzer itself (hot dialyzate). An indirect cooling of the blood (Peltier element, cooling element etc.) is also conceivable.

In an embodiment, the heating apparatus is a flow heater arranged at a line of the respective fluid system. The cooling apparatus can be a flow cooler arranged at a line of the respective fluid system.

In an embodiment, the heating apparatus comprises a heat exchanger, for example a spiral heat exchanger, a Peltier element and/or a heating pack as a heating element. The cooling apparatus can comprise a heat exchanger, for example a spiral heat exchanger, a Peltier element and/or a cooling pack as a cooling element.

In an embodiment, the dialysis device has a feed system for an anticoagulant fluid, such as a citrate solution or a heparin solution, which comprises an inlet line opening into the extracorporeal blood system at the feed side and/or at the return side of the dialyzer. A temperature control or a contribution to the temperature control can likewise be achieved using this feed system and the temperature setting of the anticoagulant fluid, such as has been explained in connection with the substitution fluid system.

To control the temperature of all fluids, temperature sensors which are connected to the control unit can be arranged at suitable points in the dialysis device. For example, a temperature sensor can be arranged before or at the dialyzer in the extracorporeal blood system to monitor the temperature of the blood before or in the dialyzer. Furthermore, a temperature sensor can be arranged in the blood system at the return side of the cooling apparatus or opening to monitor the temperature of the blood before the reinfusion to the patient. A temperature sensor can furthermore be arranged before the dialyzer in the dialyzing fluid system to monitor the temperature of the dialyzing fluid before the dialyzer. A temperature sensor can also be arranged before the opening in the pre-dilution line and/or post-dilution line of the substitution fluid system to monitor the temperature of the substitution fluid before entry into the extracorporeal blood system.

Both the heating mechanism and the cooling mechanism can combine different ones of the named corresponding mechanisms.

The temperature of the heated dialyzing fluid solution, substitution solution or anticoagulant solution or the temperature of the heating elements arranged at the blood system preferably does not exceed a temperature of 46° C. in order not to cause any local denaturation of blood components during the feeding or at the contact point.

A dialysis process can be carried out using the dialysis device in accordance with the invention, wherein the blood is heated to the dialysis temperature before entry into the dialyzer or in the dialyzer and is cooled back to the body temperature of the patient after exiting the dialyzer.

Further details and advantages of the invention result from the embodiments represented in the following with reference to the Figures. There are shown in the Figures:

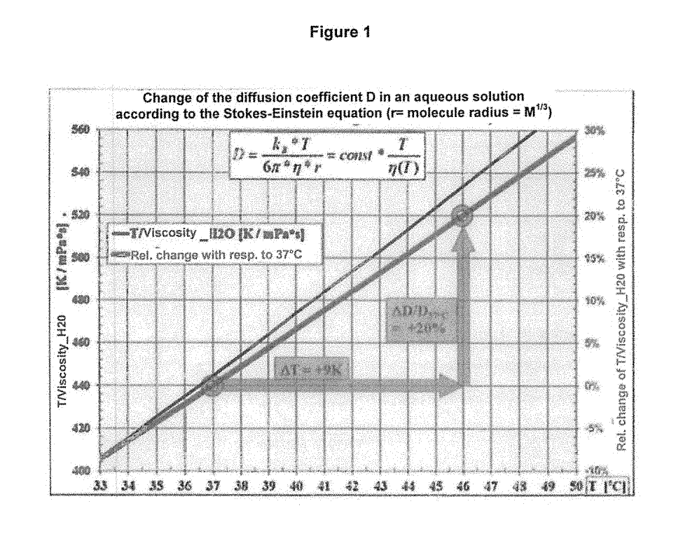

FIG. 1: a modeled representation of the change of the diffusion coefficient D in an aqueous solution according to the Stokes-Einstein equation;

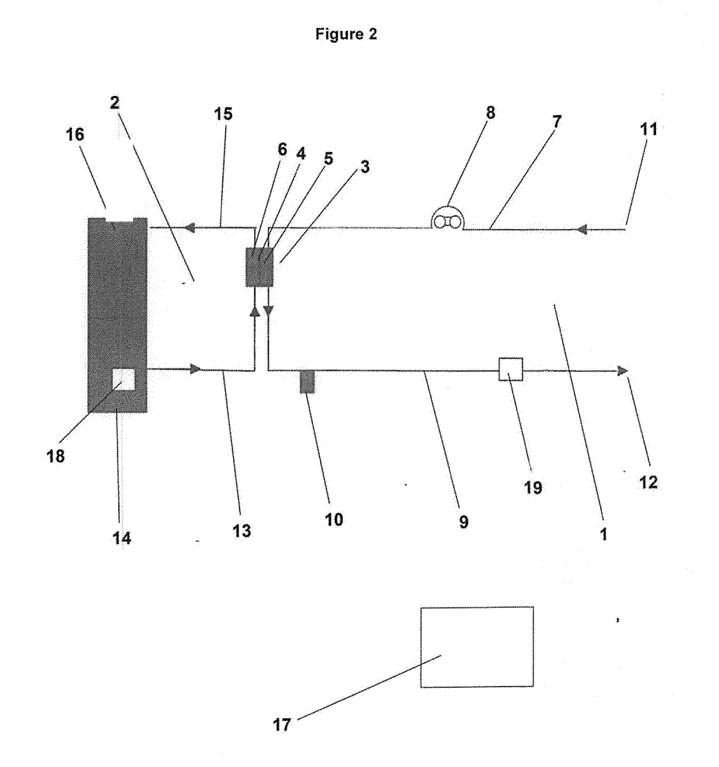

FIG. 2: a schematic representation of an embodiment of a dialysis device in accordance with the invention;

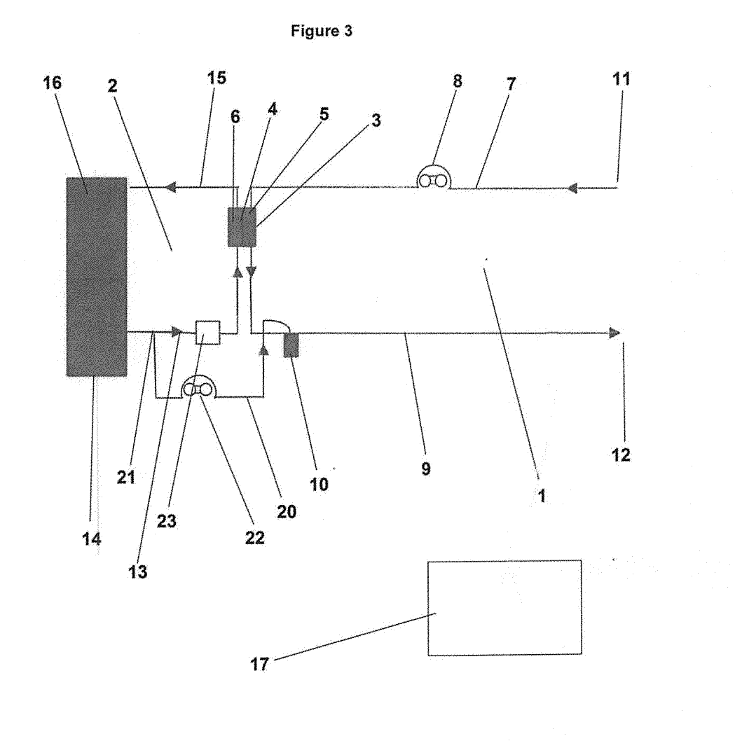

FIG. 3: a schematic representation of a further embodiment of a dialysis device in accordance with the invention;

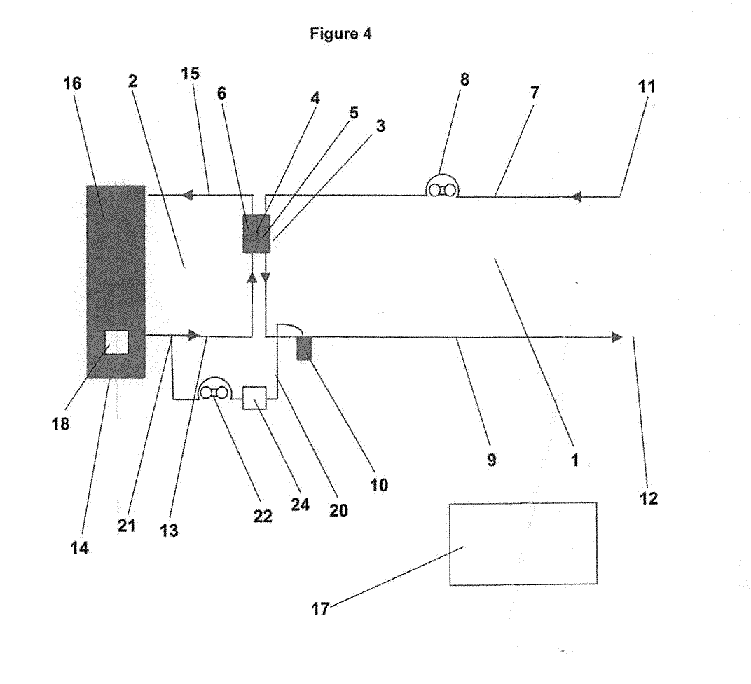

FIG. 4: a schematic representation of a further embodiment of a dialysis device in accordance with the invention; and

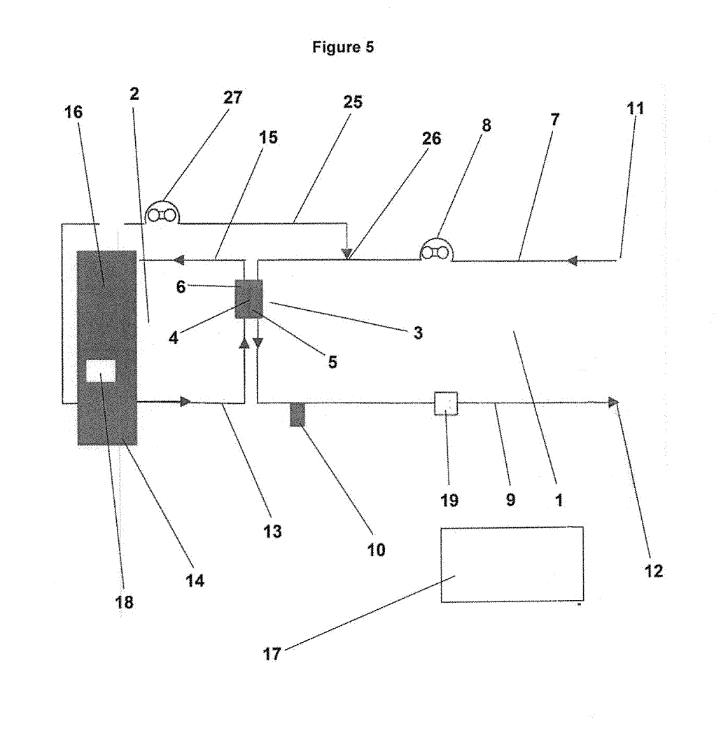

FIG. 5: a schematic representation of a further embodiment of a dialysis device in accordance with the invention.

A modeled representation of the change of the diffusion coefficient D in an aqueous solution according to the Stokes-Einstein equation is shown in FIG. 1. The Stokes-Einstein equation describes the diffusion coefficient in dependence on the temperature T, on the viscosity η of the solvent and on the radius r of the diffusing molecule.

D=k_{B}·T/6·π·η·r=const·T/η(T)

The viscosity of the water (plasma) is likewise temperature-dependent. In the representation in accordance with FIG. 1, the ratio T/η and its relative change with respect to 37° C. is shown as a function of the temperature. With a temperature increase from 37° C. to 46° C. (ΔT=+9K), an increase of the diffusion coefficient D by 20% accordingly results for all molecules.

A first embodiment of a dialysis device in accordance with the invention is shown schematically in FIG. 2.

The dialysis device comprises an extracorporeal blood circuit 1 and a dialyzing fluid circuit 2 which come into contact with one another at a dialyzer 3. The dialyzer 3 comprises a semipermeable membrane 4 which separates a blood chamber 5, which forms a part of the extracorporeal blood circuit 1, and a dialyzing fluid chamber 6, which forms a part of the dialyzing fluid circuit 2, from one another. The flow directions of the blood and of the dialyzing fluid in the different chambers 5 and 6 of the dialyzer 3 are opposite directions. The flow directions in the circuits are indicated by arrows in the Figure.

A blood pump 8 is located in the arterial blood line 7 and a drip chamber 10 is located in the venous blood line 9. The arterial port and the venous port 12 for connection to the patient are marked by the reference numerals 11 and 12.

The feed line 13 of the dialyzing fluid circuit 2 is connected to a dialyzing fluid source 14. The source can, for example, be a reservoir individual to a machine or a continuous mixing unit individual to a machine. It is furthermore conceivable that the source 14 represents a central supply unit of a dialysis center. The return line 15 of the dialyzing fluid circuit 2 is connected to a drain 16.

The dialysis device furthermore has a control unit 17 which inter alia regulates the flow rates in the extracorporeal blood circuit 1 and in the dialyzing fluid circuit 2 using the pump 8 and further actuators not shown in FIG. 2, but familiar to the skilled person.

In accordance with the invention, the dialysis device has a heating mechanism for heating the blood on passing through the dialyzer 3. In the embodiment shown, the heating mechanism comprises a heating apparatus 18 which is arranged in the source 14 or in the feed lines 13 and which (not shown in the Figure) is connected to the control unit 17. This heating apparatus 18 is controlled by the control unit 17 such that dialysis fluid is elevated to a temperature above the body temperature of the patient before it enters the dialyzing fluid chamber 6 of the dialyzer 3. The blood is continuously heated on passing through the blood chamber 5 of the dialyzer 3 by a heat exchange at the semipermeable membrane 4 until it reaches a dialysis temperature close to the venous outlet of the dialyzer 3 which is preferably above 40° C. An increased purification performance is thereby achieved at least in the venous half of the dialyzer 3 due to the previously described effects. The temperature of the dialyzing fluid entering into the dialyzer 3 can be monitored, for example, using a temperature sensor not shown in the Figure which is located in the feed line 13 of the dialyzing fluid circuit 2, preferably close to the dialyzer 3, and which is likewise connected to the control unit 17.

To cool the blood back to body temperature, which was heated in the dialyzer 3 to a dialysis temperature above the body temperature of the patient, before reinfusion into the patient at the venous port 12, the dialysis device furthermore has a cooling mechanism which comprises a heat exchanger 19 in the venous line 9. The heat exchanger 19 can, for example, be configured as a spiral heat exchanger, wherein the blood is brought into heat-conductive contact with a cooling fluid which has a temperature below body temperature. The heat exchanger or the fluid pump for the cooling fluid is likewise connected to the control unit 17. To monitor the blood temperature before reinfusion at the venous port 12, a temperature sensor which is not shown in the Figure and which is likewise connected to the control unit 17 can be present in the venous line 9, preferably close to the venous port 12 and in any case between the heat exchanger 19 and the venous port 12.

A further embodiment of a dialysis device in accordance with the invention is shown in FIG. 3, with the same parts being provided with the same reference numerals.

In this respect, the cooling mechanism comprises a post-dilution line 20 which branches off from the feed line 13 at a point 21. A fluid pump 22 is arranged within the post-dilution line. This post-dilution line 20 opens into the venous line 9 at the drip chamber 10.

In this embodiment, a heating apparatus 23 is provided in the feed line 13 between the branching point 21 and the dialyzer 3. It is thus possible to further increase the temperature of the dialyzing fluid which is supplied to the dialyzer 3 after the branching off of the substitution solution for the post-dilution. Provision can be made to this extent that the dialyzing fluid is not heated or is at least not heated up to body temperature during the provision in the source 14 and is branched off into the post-dilution line 20 at the branching point 21 in this still cool state. An increase of the temperature to above body temperature only takes place between the branching point 21 and the dialyzer 3 using the heating apparatus 23 so that the heating effects of the blood are adopted in the dialyzer 3 which have already been shown in connection with the embodiment in accordance with FIG. 1.

The heating apparatus 23 and the pump 22 are connected to the control unit 17.

In contrast to the apparatus in accordance with FIG. 1, the cooling of the blood after leaving the dialyzer 3 does not take place by an addition cooling unit 19, but rather by the supply of substitution solution which has a temperature which is below body temperature. The advantage of this solution is that no heating, element and/or cooling element is required at the extracorporeal blood circuit 1.

A further embodiment of the invention is shown in FIG. 4, wherein the same parts are again marked by identical reference numerals. This embodiment is similar to the embodiment in accordance with FIG. 3 with the sole difference that a cooling apparatus 24 is arranged in the post-dilution line 20 instead of the heating apparatus 23 in the fed line 13. The dialyzing fluid is thus, as was also the case in the embodiment in accordance with FIG. 2, already increased above body temperature already during the provision and is also branched off at the branching point 21 into the post-dilution line 20 also at an elevated temperature. The dialyzing fluid or substitution fluid is subsequently cooled by the cooling apparatus 24 in the post-dilution line 20 before it is introduced into the venous line 9 of the extracorporeal blood circuit 1. This embodiment has the advantage with respect to the embodiment in accordance with FIG. 3 that an improved regulating capability of the cooling power is present by using the selective cooling of the substitution fluid in the post-dilution line 20.

The cooling apparatus 24, the heating apparatus 18 and the pump 22 are connected to the control unit 17.

In the embodiment in accordance with FIGS. 3 and 4, a temperature sensor can be present in the post-dilution line 20, preferably close to the drip chamber 10, to be able to determine the temperature of the substitution solution to be fed into the venous line 9. This temperature sensor is in turn connected to the control unit 17.

FIG. 5 shows a further embodiment of a dialysis device in accordance with the invention, wherein the difference between the embodiment in accordance with FIG. 2 lies in the fact that a pre-dilution line 25 is provided by which substitution solution is branched off from the source 14 and is admixed Into the arterial line 7 at a mixing point 26 between the pump 8 and the dialyzer 3.

A pump 27 is located in the pre-dilution line 25. There is the possibility in this embodiment to introduce substitution fluid heated in the source 14 to a temperature above the body temperature of the patient into the arterial line 7 and thus already to heat the blood to a dialysis temperature of, for example, more than 40° C. before entry into the dialyzer 3. The treatment efficiency can be increased to this extent over the total dialyzer in this manner. A further heating by the use of a dialyzing fluid heated above body temperature at the dialyzing fluid side 6 of the dialyzer 3 is additionally possible.

Provision is preferably made in this embodiment to provide a temperature sensor close to the injection point 26 in the pre-dilution line 25 and to connect said temperature sensor to the control unit 17 to be able to monitor the temperature of the injected substitution solution. The pump 27 is likewise connected to the control unit 17.

1. A dialysis device comprising an extracorporeal blood system, a dialyzing fluid system, a dialyzer and a control unit, characterized in that the dialysis device has a heating mechanism for heating the blood in the extracorporeal blood system before entry into the dialyzer or in the dialyzer as well as a cooling mechanism for cooling the blood in the extracorporeal blood system after exiting the dialyzer; and in that the control unit is configured such that the blood is heated before entry into the dialyzer or in the dialyzer to a dialysis temperature which is above the body temperature of the patient and is cooled back to the body temperature of the patient after exiting the dialyzer.

2. A dialysis device in accordance with claim 1, characterized in that the dialysis temperature is between 37° C. and 46° C., preferably between 40° C. and 46° C., and further preferably between 42° C. and 45° C.

3. A dialysis device in accordance with claim 1, characterized in that the heating mechanism comprises a heating apparatus arranged at the feed side of the dialyzer in the dialyzing fluid system; and in that the control unit is configured such that the dialyzing fluid is heated before entry into the dialyzer to a temperature which is larger than or at least equal to the dialysis temperature.

4. A dialysis device in accordance with claim 1, characterized in that the dialysis device furthermore has a substitution fluid system which comprises a pre-dilution line opening at the feed side of the dialyzer into the extracorporeal blood system and/or a post-dilution line opening at the return side of the dialyzer into the extracorporeal blood system.

5. A dialysis device in accordance with claim 4, characterized in that the heating mechanism comprises a heating apparatus arranged at the feed side of the opening of the pre-dilution line in the substitution fluid system; and in that the control unit is configured such that the substitution fluid is heated before entry into the extracorporeal blood system through the pre-dilution line to a temperature which is larger than or at least equal to the dialysis temperature.

6. A dialysis device in accordance with claim 5, characterized in that the cooling mechanism comprises a cooling apparatus arranged at the feed side of the opening of the post-dilution line in the substitution fluid system; and in that the control unit is configured such that the substitution fluid is cooled before entry into the extracorporeal blood system through the post-dilution line to a temperature which is less than or at a maximum equal to body temperature.

7. A dialysis device in accordance with claim 5, characterized in that the cooling mechanism has a branch for substitution fluid arranged at the feed side of the heating apparatus in the substitution fluid system, the temperature of the substitution fluid being less than or at a maximum equal to body temperature; and in that the control unit is configured such that the substitution fluid is branched off before entry into the heating apparatus and is not heated up to body temperature through the post-dilution line before entry into the extracorporeal blood system.

8. A dialysis device in accordance with claim 1, characterized in that the heating mechanism comprises a heating apparatus arranged at the feed side of the dialyzer in the blood system; and/or in that the cooling mechanism comprises a cooling apparatus arranged at the return side of the dialyzer in the blood system.

9. A dialysis device in accordance with claim 3, characterized in that the heating apparatus is a flow heater arranged at a line of the respective fluid system; and/or in that the heating apparatus comprises a heat exchanger, for example a spiral heat exchanger, a Peltier element and/or a heating pack as a heating element.

10. A dialysis device in accordance with claim 3, characterized in that the cooling apparatus is a flow cooler arranged at a line of the respective fluid system; and/or in that the cooling apparatus comprises a heat exchanger, for example a spiral heat exchanger, a Peltier element and/or a cooling pack as a cooling element.
